(19) United States
(12) Patent Application Publication
(10) Pub. No.: US 2006/0020276 A1
(43) Pub. Date: Jan. 26, 2006

(54) Apparatus and methods for achieving prolonged maintenance of gastrointestinal tissue folds

(75) Inventors: Vahid Saadat, Saratoga, CA (US); Desmond Birkett, Boston, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 10/898,683

(22) Filed: Jul. 23, 2004

(51) Int. Cl. A61B 17/08

(52) U.S. Cl. 606/153

(57) ABSTRACT

Apparatus and methods are provided for achieving prolonged maintenance of gastrointestinal (“GI”) tissue folds. The apparatus and methods are adapted to form, secure, approximate and promote healing of tissue folds. Such healing may comprise remodeling the tissue folds along region(s) of opposing tissue contact to facilitate prolonged maintenance of the folds. In one variation, the apparatus and methods may be utilized to achieve gastric reduction. The apparatus may be provided as a system of tools.

### BACKGROUND OF THE INVENTION

### FIELD OF THE INVENTION

The present invention relates to methods and apparatus for achieving prolonged maintenance of gastrointestinal (“GI”) tissue folds by forming, securing and promoting healing of the folds. More particularly, the present invention provides methods and apparatus for remodeling the tissue folds to facilitate their prolonged maintenance.

Extreme or morbid obesity is a serious medical condition pervasive in the United States and other countries. Its complications include hypertension, diabetes, coronary artery disease, stroke, congestive heart failure, multiple orthopaedic problems and pulmonary insufficiency with markedly decreased life expectancy.

Several surgical techniques have been developed to treat morbid obesity, including bypassing an absorptive surface of the small intestine, bypassing a portion of the stomach, and reducing or partitioning the stomach size, e.g., via Vertical Banded Gastroplasty (“VBG”) or Magenstrasse and Mill. These procedures may be difficult to perform in morbidly obese patients and/or may present numerous potentially life-threatening post-operative complications. Thus, less invasive techniques have been pursued.

U.S. Pat. Nos. 4,416,267 and 4,485,805 to Garren et al. and Foster, Jr., respectively, propose insertion of an inflated bag within a patient's stomach to decrease the effective volume of the stomach that is available to store food. Accordingly, the patient is satiated without having to consume a large amount of food. A common problem with these inflated bags is that, since the bags float freely within the patient's stomach, the bags may migrate to, and block, a patient's pyloric opening, the portal leading from the stomach to the duodenum, thereby restricting passage of food to the remainder of the gastro-intestinal tract.

Apparatus and methods also are known in which an adjustable elongated gastric band is laparoscopically disposed around the outside of a patient's stomach near the esophagus to form a collar that, when tightened, squeezes the stomach into an hourglass shape, thereby providing a stoma that limits the amount of food that a patient may consume comfortably. An example of an adjustable gastric band is the LAP-BAND® made by INAMED Health of Santa Barbara, Calif.

Numerous disadvantages are associated with using an adjustable gastric band. First, the band may be dislodged if the patient grossly overeats, thereby requiring additional invasive surgery to either reposition or remove the band. Similarly, overeating may cause the band to injure the stomach wall if the stomach over-expands. Laparoscopic disposal of the gastric band around the stomach requires a complex procedure, requires considerable skill on the part of the clinician, and is not free of dangerous complications.

In view of the drawbacks associated with prior art techniques for treating morbid obesity, Applicant previously has described methods and apparatus for treating obesity by endoscopically forming and approximating tissue folds within a patient's stomach in order to reduce an effective cross-section of the stomach. See, for example, co-pending U.S. patent applications Ser. No. 10/735,030, filed Dec. 12, 2003, and Ser. No. TO BE ASSIGNED [Attorney Docket No. 021496-000800US], filed May 7, 2004, both of which are incorporated herein be reference in their entireties. Those references describe formation of serosa-to-serosa gastric plications from the interior of the stomach, such that mucosal tissue is in contact with opposing mucosal tissue along the interior of each gastric tissue fold.

Mucosal tissue typically will not fuse or heal together with opposing, contacting mucosal tissue. Thus, gastric tissue folds may require securing elements, such as anchors, clips or suture, to maintain the folds for prolonged periods of time. If such securing elements were ever to be removed or to ever fail, the gastric tissue folds or plications might unfold.

U.S. patent Publication Ser. No. 2004/0034371 to Lehman et al., published Feb. 19, 2004 (Ser. No. 10/275,521, PCT filed May 18, 2001), which is incorporated herein by reference in its entirety, describes methods and devices for promoting tissue adhesion. The healing process is utilized to form scar tissue that bonds two tissue surfaces together. A tissue injury is accomplished by destroying the mucosal layer of tissue. After the injury is initiated, the injured tissue surfaces are held in close contact, and, as scar tissue created by the injury forms, the tissue surfaces become bonded together in a permanent union. The Lehman reference does not describe removing at least a portion of the injured tissue prior to tissue bonding, nor does it describe using shape-lockable tools to access and/or manipulate the tissue surfaces.

In view of the foregoing, it would be desirable to provide methods and apparatus for achieving prolonged maintenance of gastrointestinal tissue folds without necessitating prolonged use of securing elements.

It would be desirable to provide methods and apparatus that promote remodeling of the folds to facilitate their prolonged maintenance.

### BRIEF SUMMARY OF THE INVENTION

Prolonged maintenance of gastrointestinal (“GI”) tissue folds is achieved by providing methods and apparatus for forming, securing and promoting healing of the folds. Such healing may comprise remodeling the tissue folds along region(s) of opposing tissue contact to facilitate prolonged maintenance of the folds. For example, opposing mucosal tissue in contact along each fold may be ablated, abraded, burnt, charred, cut, chemically irritated, biologically irritated or otherwise injured, and preferably removed, to initiate a wound healing response that remodels the tissue and results in scar tissue formation with concomitant fusion of each fold at region(s) of opposing mucosal tissue contact. Such injury and/or removal of mucosal tissue may, for example, be achieved via electromagnetic, thermal, chemical, biologic or mechanical modalities, or a combination thereof. Additional injury/removal modalities will be apparent to those of skill in the art.

During the period of time necessary for remodeling/scar tissue formation, securing elements may be utilized to maintain, e.g., temporarily maintain, contact of opposing injured tissue along each tissue fold. Such securing elements optionally may be removed or may biodegrade upon formation of scar tissue. Alternatively, the elements may be left in place indefinitely to reinforce the scar tissue and ensure prolonged maintenance of the tissue folds. However, upon formation of the scar tissue, the securing elements may no longer solely maintain the folds.

In one aspect, an injury tool is provided to locally injure mucosal tissue. The injury tool may comprise, for example, an ablation tool, such as a monopolar or bipolar Radio Frequency (“RF”) ablation tool. Alternatively, the injury tool may comprise a mechanical abrasion tool. Additional injury tools will be apparent to those of skill in the art.

In one aspect, the injury tool may comprise suction to facilitate tissue engagement and/or aspiration. In another aspect, the tool comprises fluid injection for tissue cooling or cleansing. In yet another aspect, the tool may comprise a removal element for removing injured tissue. Removed tissue may be captured or allowed to pass through the GI tract. The injury tool also may optionally comprise a depth-limiting element that limits a depth of tissue injury.

The injury tool preferably is used in combination with additional tools, e.g., for forming and securing the gastrointestinal tissue folds. These tools may comprise tools for accessing, visualizing, grasping, maneuvering, piercing, folding, plicating, approximating, securing, characterizing, sampling and/or suturing GI tissue. One or more of the tools optionally may be steerable or shape-lockable/rigidizable. Additional tools will be apparent to those of skill in the art. Such tools may be provided as a kit or system in combination with the injury tool. Furthermore, the injury tool may be integrated into a multi-functional tool.

Methods of using apparatus of the present invention also are provided.

### BRIEF DESCRIPTION OF THE DRAWINGS

FIGS. 1A-1C are, respectively, a side view, a detail side view and a detail perspective view of one variation of a tissue plication apparatus that may be used to create tissue plications and to deliver securing elements into the tissue.

FIGS. 2A-2D are detail side views, partially in section, illustrating a method of utilizing the apparatus of FIG. 1 to plicate or fold tissue, and to secure the folded tissue via tissue securing elements.

FIGS. 3A-3G are detail cross-sectional views of apparatus for delivering exemplary tissue anchor securing elements, illustrating a method of maintaining a tissue plication via anchors disposed on proximal and distal sides of the plication.

FIG. 4 is a detail cross-sectional view illustrating approximation and securement of multiple, e.g., opposing, tissue folds.

FIG. 5 is a detail cross-sectional view illustrating remodeling and fusion of approximated tissue folds.

FIG. 6 is a cross-sectional view illustrating an endoluminal gastric reduction procedure achievable through remodeling of approximated tissue folds.

FIG. 7 is a schematic perspective view of one variation of an injury tool comprising an ablation tool.

FIGS. 8A and 8B are detail views, partially in section, illustrating variations on a method of using the injury tool of FIG. 7 in combination with the apparatus of FIGS. 1-3 to fold, secure, approximate and promote healing or remodeling of tissue folds.

FIGS. 9A-9F are schematic views of multiple variations of the injury tool.

FIGS. 10A and 10B are schematic views of additional variations of the injury tool comprising a mechanical injury element.

### DETAILED DESCRIPTION OF THE INVENTION

Methods and apparatus for achieving prolonged maintenance of gastrointestinal (“GI”) tissue folds by forming, securing and promoting healing of the folds are described below. The methods and apparatus may be utilized within the GI system of a patient in various ways, e.g., transorally, endoluminally, percutaneously, laparoscopically, etc. More particularly, methods and apparatus for remodeling approximated tissue folds to facilitate their prolonged maintenance are disclosed.

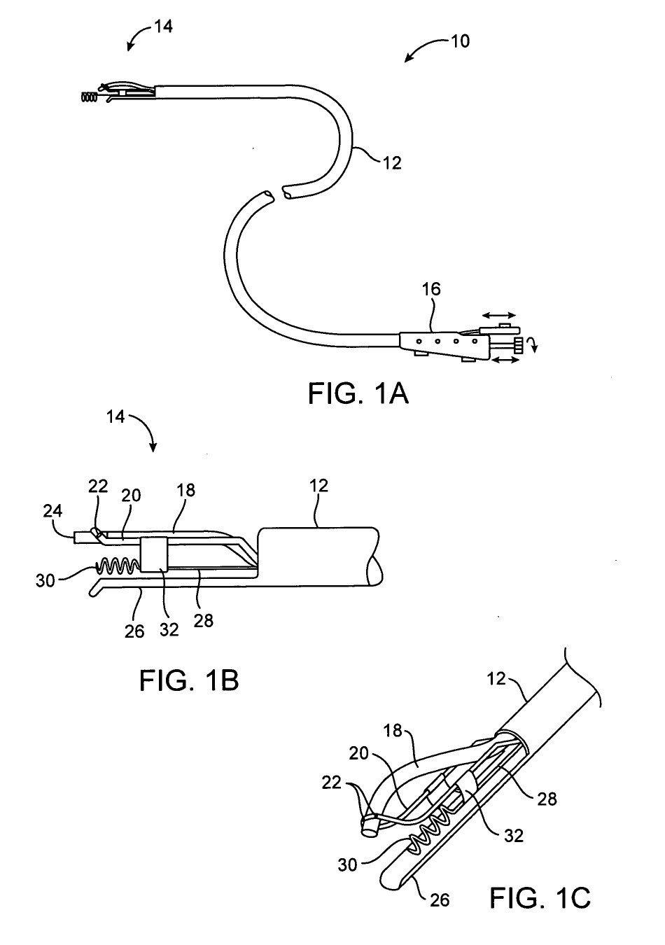

With reference to FIGS. 1-4, in order to form and secure one or more plications or tissue folds within a body lumen of a patient, various methods and devices may be implemented. For example, securing elements, such as suture, anchors and/or clips, as well as assemblies formed therefrom, may be delivered and positioned via an endoscopic apparatus that engages a tissue wall of the gastrointestinal lumen, creates one or more tissue folds, and disposes one or more of the elements through the tissue fold(s). The securing element(s) may be disposed through the muscularis and/or serosa layers of the gastrointestinal lumen. The endoscopic apparatus may also approximate and secure multiple folds together.

Generally, in creating a plication through which a securing element may be disposed within or through, a distal tip of a tissue plication apparatus may engage or grasp the tissue and move the engaged tissue to a proximal position relative to the tip of the device, thereby providing a substantially uniform plication of predetermined size.

Formation of a tissue fold may, for example, be accomplished using at least two tissue contact areas that are separated by a linear or curvilinear distance, wherein the separation distance between the tissue contact points affects the length and/or depth of the fold. In operation, a tissue grabbing assembly engages or grasps the tissue wall in its normal state (i.e., non-folded and substantially flat or non-plicated), thus providing a first tissue contact area. The first tissue contact area then is moved to a position proximal of a second tissue contact area to form the tissue fold. The tissue securing element(s) then may be extended across the tissue fold at the second tissue contact area. Optionally, a third tissue contact point may be established such that, upon formation of the tissue fold, the second and third tissue contact areas are disposed on opposing sides of the tissue fold, thereby providing backside stabilization during extension of the securing element(s) across the tissue fold from the second tissue contact area.

The first tissue contact area may be utilized to engage and then stretch or rotate the tissue wall over the second tissue contact area to form the tissue fold. The tissue fold may then be articulated to a position where a portion of the tissue fold overlies the second tissue contact area at an orientation that is substantially normal to the tissue fold. A tissue securing element may then be delivered across the tissue fold at or near the second tissue contact area. An apparatus which is particularly suited to deliver the securing elements described herein may be seen in further detail in co-pending U.S. patent application Ser. No. 10/735,030 filed Dec. 12, 2003 and entitled “Apparatus And Methods For Forming And Securing Gastrointestinal Tissue Folds,” which previously has been incorporated herein by reference.

An illustrative side view of a tissue plication assembly 10 which may be utilized with tissue securing elements described herein is shown in FIG. 1A. The plication assembly 10 generally comprises a catheter or tubular body 12 which may be configured to be sufficiently flexible for advancement into a body lumen, e.g., transorally, endoluminally, percutaneously, laparoscopically, etc. Tubular body 12 may be configured to be torqueable through various methods, e.g., utilizing a braided tubular construction, such that when handle 16 is manipulated and rotated by a practitioner from outside the body, the torquing force is transmitted along body 12 such that the distal end of body 12 is rotated in a corresponding manner.

Tissue manipulation assembly 14 is located at the distal end of tubular body 12 and is generally used to contact and form the tissue plication, as mentioned above. FIG. 1B shows an illustrative detail side view of tissue manipulation assembly 14 which shows launch tube 18 extending from the distal end of body 12 and in-between the arms of upper extension member or bail 20. Launch tube 18 may define launch tube opening 24 and may be pivotally connected near or at its distal end via hinge or pivot 22 to the distal end of upper bail 20. Lower extension member or bail 26 may similarly extend from the distal end of body 12 in a longitudinal direction substantially parallel to upper bail 20. Upper bail 20 and lower bail 26 need not be completely parallel so long as an open space between upper bail 20 and lower bail 26 is sufficiently large enough to accommodate the drawing of several layers of tissue between the two members.

Upper bail 20 is shown in the figure as an open looped member and lower bail 26 is shown as a solid member; however, this is intended to be merely illustrative and either or both members may be configured as looped or solid members. Tissue acquisition member 28 may be an elongate member, e.g., a wire, hypotube, etc., which terminates at a tissue grasper 30, in this example a helically-shaped member, configured to be reversibly rotatable for advancement into the tissue for the purpose of grasping or acquiring a region of tissue to be formed into a plication. Tissue acquisition member 28 may extend distally from handle 16 through body 12 and distally between upper bail 20 and lower bail 26. Acquisition member 28 may also be translatable and rotatable within body 12 such that tissue grasper 30 is able to translate longitudinally between upper bail 20 and lower bail 26. To support the longitudinal and rotational movement of acquisition member 28, an optional guide or sled 32 may be connected to upper bail 20 or lower bail 26 to freely slide thereon. Guide 32 may also be slidably connected to acquisition member 28 such that guide 32 supports the longitudinal motion of acquisition member 28.

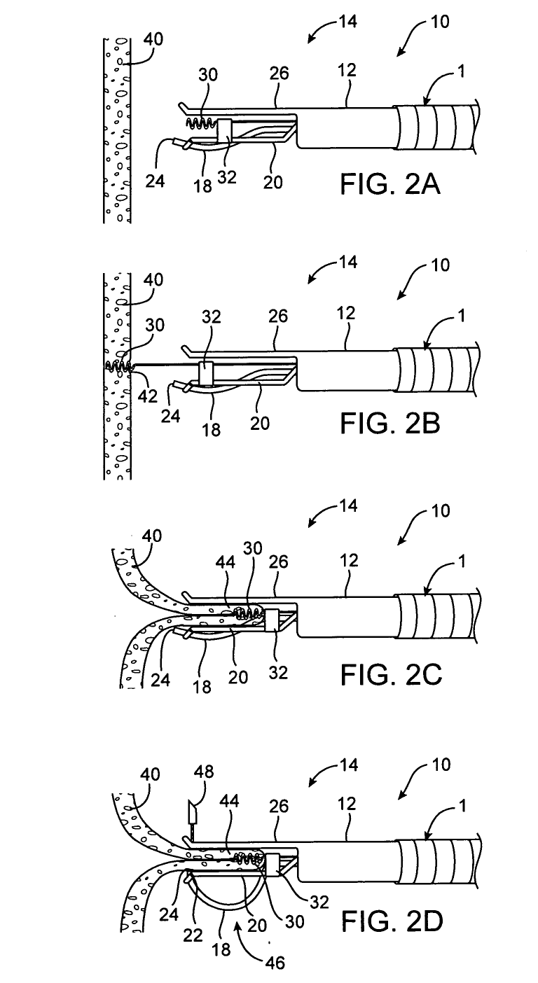

An example of a tissue plication procedure for forming and securing a tissue fold, illustratively with a securing element comprising a tissue anchor, is seen in FIGS. 2A-2D and is disclosed in further detail in co-pending U.S. patent application Ser. No. 10/735,030 filed Dec. 12, 2003, which has been incorporated by reference above. As seen in FIG. 2A, tissue manipulation assembly 14 of plication assembly 10 may be advanced into a body lumen such as the stomach and positioned adjacent to a region of tissue wall 40 to be plicated.

Plication assembly 10 illustratively has been advanced through steerable and/or shape-lockable or rigidizable overtube 1 to facilitate positioning of tissue manipulation assembly 14 adjacent the desired region of tissue wall 40. Furthermore, overtube 1 aids force transmission along plication assembly 10 to facilitate tissue folding, securing, approximating, etc. Exemplary steerable and/or shape-lockable overtubes are described in greater detail in Applicant's co-pending U.S. patent application Ser. No. TO BE ASSIGNED [Attorney Docket No. 021496-000130US], filed Mar. 9, 2004, and entitled, “Endoluminal Tool Deployment System,” which is incorporated herein by reference in its entirety. During advancement of plication assembly 10 through overtube 1, launch tube 18 may be configured in a delivery profile such that tube 18 is disposed within or between the arms of upper bail 20 to present a relatively small profile.

Once tissue manipulation assembly 14 has been desirably positioned relative to tissue wall 40, tissue acquisition member 30 may be advanced distally such that tissue acquisition member 30 comes into contact with tissue wall 40 at acquisition location or point 42. As acquisition member 30 is distally advanced relative to body 12, guide 32, if utilized, may slide distally along with member 30 to aid in stabilizing the grasper. If a helically-shaped acquisition member 30 is utilized, as illustrated in FIG. 2B, it may be rotated from its proximal end at handle 16 and advanced distally until the tissue at point 42 has been firmly engaged by acquisition member 30. This may require advancement of acquisition member 30 through the mucosal layer and at least into or through the underlying muscularis layer and preferably into or through the serosa layer.

The grasped tissue may then be pulled proximally between upper 20 and lower bails 26 via acquisition member 30 such that the acquired tissue is drawn into a tissue fold 44, as seen in FIG. 2C. As acquisition member 30 is withdrawn proximally relative to body 12, guide 32 may also slide proximally to aid in stabilizing the device especially when drawing the tissue fold 44.

Once the tissue fold 44 has been formed, launch tube 18 may be advanced from its proximal end at handle 16 such that a portion 46 of launch tube 18, which extends distally from body 12, is forced to rotate at hinge or pivot 22 and reconfigure itself such that portion 46 forms a curved or arcuate shape that positions launch tube opening 24 perpendicularly relative to a longitudinal axis of body 12 and/or bail members 20, 26. Launch tube 18, or at least portion 46 of launch tube 18, is preferably fabricated from a highly flexible material or it may be fabricated, e.g., from Nitinol tubing material which is adapted to flex, e.g., via circumferential slots, to permit bending. Alternatively, assembly 14 may be configured such that launch tube 18 is reconfigured simultaneously with the proximal withdrawal of acquisition member 30 and acquired tissue 44.

The tissue wall of a body lumen, such as the stomach, typically comprises an inner mucosal layer, connective tissue, the muscularis layer and the serosa layer. To obtain a durable purchase, e.g., in performing a stomach reduction procedure, the securing elements, e.g., staples or anchors, used to achieve reduction of the body lumen are preferably engaged at least through or at the muscularis tissue layer, and more preferably, the serosa layer. Advantageously, stretching of tissue fold 44 between bail members 20, 26 permits an anchor to be ejected through both the muscularis and serosa layers, thus enabling durable gastrointestinal tissue approximation.

As shown in FIG. 2D, once launch tube opening 24 has been desirably positioned relative to the tissue fold 44, needle assembly 48 may be advanced through launch tube 18 via manipulation from its proximal end at handle 16 to pierce preferably through a dual serosa layer through tissue fold 44. Needle assembly 48 is preferably a hollow tubular needle through which one or several tissue anchors may be delivered through and ejected from in securing the tissue fold 44, as further described below.

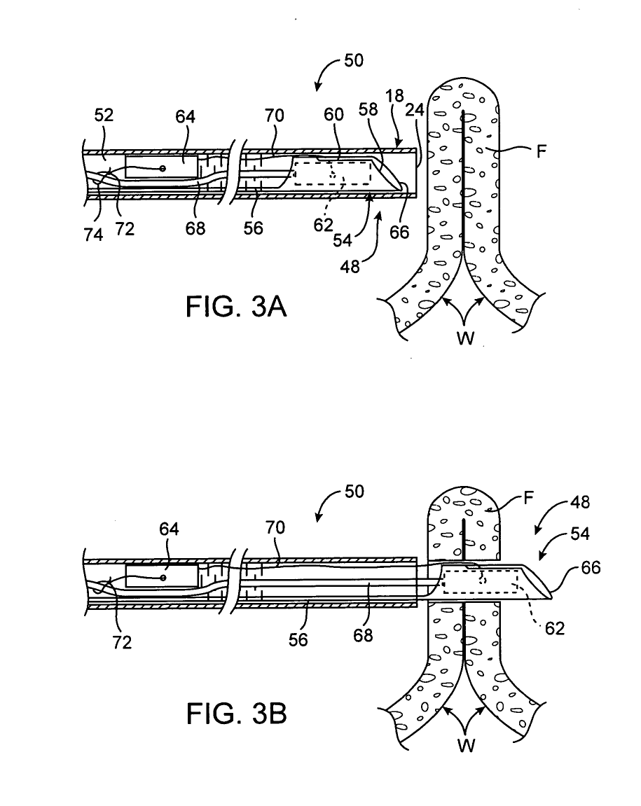

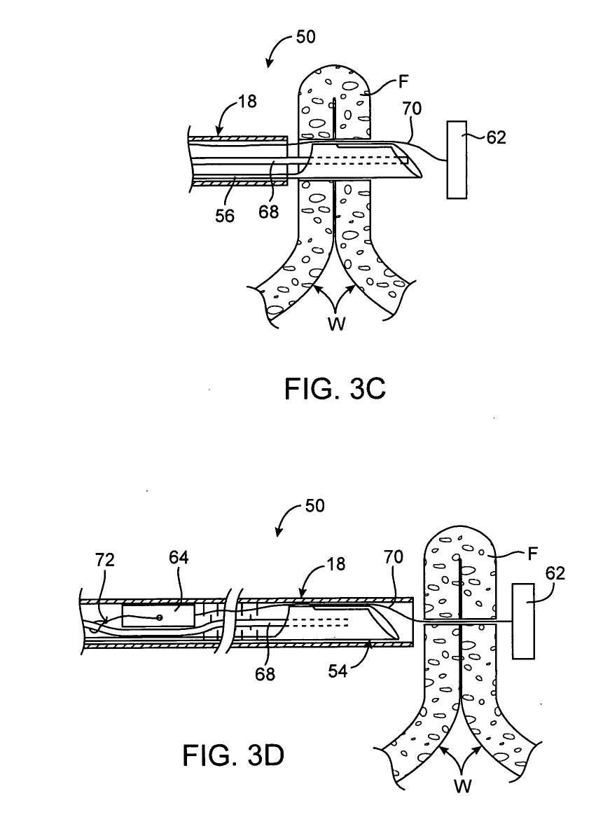

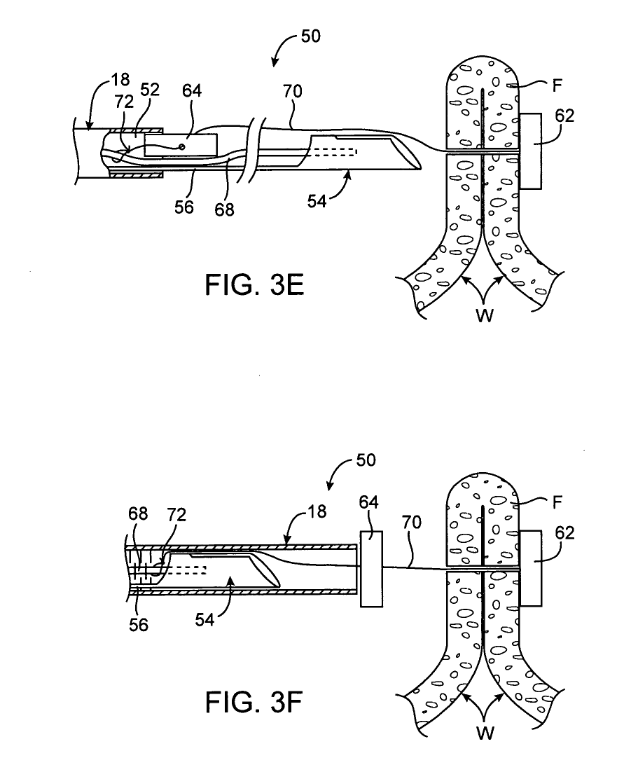

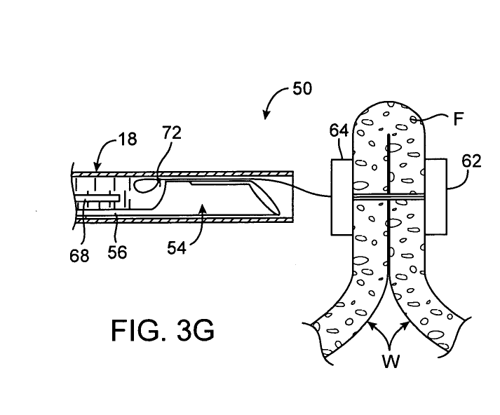

Because needle assembly 48 penetrates the tissue wall twice, it exits within the body lumen, thus reducing the potential for injury to surrounding organs. A detail cross-sectional view is shown in FIG. 3A of anchor delivery assembly 50 in proximity to tissue fold F. In this example, tissue fold F may comprise a plication of tissue created using the apparatus 10 described herein or any other tool configured to create such a tissue plication. Tissue fold F may be disposed within a gastrointestinal lumen, such as the stomach, where tissue wall W may define the outer or serosal layer of the stomach. Anchor delivery assembly 50 may generally comprise launch tube 18 and needle assembly 48 slidingly disposed within launch tube lumen 52. Needle assembly 48 is generally comprised of needle 54, which is preferably a hollow needle having a tapered or sharpened distal end 66 to facilitate its travel into and/or through the tissue. Other parts of the assembly, such as upper and lower bail members 20, 26, respectively, and tissue acquisition member 28 have been omitted from these figures only for clarity.

Once launch tube 18 has been desirably positioned with respect to tissue fold F, needle 54 may be urged or pushed into or through tissue fold F via needle pushrod or member 56 from its proximal end preferably located within handle 16. Needle 54 may define needle lumen 58 within which distal anchor 62 and/or proximal anchor 64 may be situated during deployment and positioning of the assembly. A single suture or flexible element 70 (or multiple suture elements) may connect proximal anchor 64 and distal anchor 62 to one another. For instance, element 70 may comprise various materials such as monofilament, multifilament, or any other conventional suture material, elastic or elastomeric materials, e.g., rubber, etc. Biocompatible metals may also be utilized for suture materials.

Needle 54 may optionally define needle slot 60 along its length to allow suture 70 to pass freely within and out of needle 54 when distal anchor 62 is ejected from needle lumen 58. Alternatively, rather than utilizing needle slot 60, needle 54 may define a solid structure with suture 70 being passed into needle lumen 58 via the distal opening of needle 54.

The proximal end of suture 70 may pass slidingly through proximal anchor 64 to terminate in suture loop 74 via cinching knot 72. Suture loop 74 may be omitted and the proximal end of suture 70 may terminate proximally of the apparatus 10 within control handle 16, proximally of control handle 16, or at some point distally of control handle 16. In this variation, suture loop 74 may be provided to allow for a grasping or hooking tool to temporarily hold suture loop 74 for facilitating the cinching of proximal 64 and distal 62 anchors towards one another for retaining a configuration of tissue fold F, as described in further detail below. Cinching knot 72 may also comprise a slidable knot which may be slid distally along suture 70 to lock or hold against proximal anchor 64 once the tissue fold F and anchors 62, 64 have been desirably positioned and tensioned, as also described below in further detail.

After needle assembly 48 has been pushed distally out through launch tube opening 24 and penetrated into and/or through tissue fold F, as shown in FIG. 3B, anchor pushrod or member 68 may be actuated also via its proximal end to eject distal anchor 62, as shown in FIG. 3C. Once distal anchor 62 has been ejected distally of tissue fold F, FIG. 3D shows how needle 54 may be retracted back through tissue fold F by either retracting needle 54 back within launch tube lumen 52 or by withdrawing the entire anchor delivery assembly 50 proximally relative to tissue fold F.

FIG. 3E shows that once needle 54 has been retracted, proximal anchor 64 may then be ejected from launch tube 18 on a proximal side of tissue fold F. With both anchors 62, 64 disposed externally of launch tube 18 and suture 70 connecting the two, proximal anchor 64 may be held against the distal end of launch tube 18 and urged into contact against tissue fold F, as shown in FIGS. 3F and 3G, respectively. As proximal anchor 64 is urged against tissue fold F, proximal anchor 64 or a portion of suture 70 may be configured to provide any number of directionally translatable locking mechanisms which provide for movement of an anchor along suture 70 in a first direction and preferably locks, inhibits, or prevents the reverse movement of the anchor back along suture 70. In other alternatives, the anchors may simply be delivered through various elongate hollow tubular members, e.g., a catheter, trocars, etc.

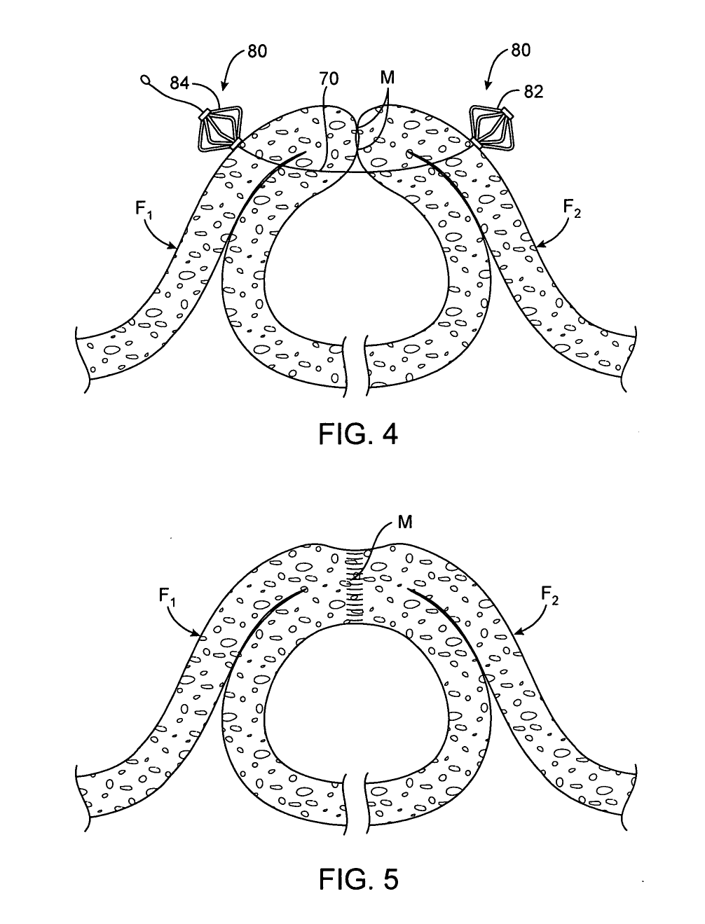

Referring now to FIG. 4, it may be desirable to approximate multiple tissue folds, such as posterior and anterior folds within a patient's stomach to partition the stomach and create a restriction that limits the passage of food therethrough. FIG. 4 illustrates approximation of fold F₁ and fold F₂. The folds illustratively are secured with a securing element comprising basket-type anchor assembly 80 having distal basket 82 and proximal basket 84 connected by suture 70. When folds F₁ and F₂ comprise gastrointestinal tissue folds formed from the interior of the GI lumen, upon approximation of the tissue folds, the folds contact each other along adjacent regions of mucosal tissue M. However, the mucosal tissue typically will not heal together along the contacting tissue interface. Thus, a securing element, such as anchor assembly 80, may be required to maintain approximation of folds F₁ and F₂.

FIGS. 1-4 have illustrated exemplary methods and apparatus for forming, securing and approximating tissue folds. However, these methods and apparatus are provided only for the sake of illustration and in no way should be construed as limiting. Furthermore, with respect to the securing elements and anchor assemblies described herein, the types of anchors shown and described are intended to be illustrative and are not limited to the variations shown. For instance, “T”-type tissue anchors have been shown, as well as reconfigurable “basket”-type anchors, which may generally comprise a number of configurable struts or legs extending between at least two collars or support members. Other variations of these or other types of anchors are also contemplated for use in securing tissue folds. Examples of such securing elements are disclosed in co-pending U.S. patent application Ser. No. 10/612,170, filed Jul. 1, 2003, which is incorporated herein by reference in its entirety. Moreover, a single type of anchor may be used exclusively in an anchor assembly; alternatively, a combination of different anchor types may be used in an anchor assembly. Furthermore, the different types of cinching or locking mechanisms are not intended to be limited to any of the particular variations shown and described but may be utilized in any of the combinations or varying types of anchors as practicable.

With reference now to FIG. 5, achieving prolonged maintenance of approximated tissue folds by promoting healing of the folds along the regions of mucosal contact is preferably achieved utilizing methods and apparatus described herein. Such healing may comprise remodeling the tissue folds. For example, opposing mucosal tissue in contact along each fold may be abraded, burnt, ablated, charred, irritated or otherwise injured, and preferably removed, to initiate a wound healing response that remodels the tissue and results in scar tissue formation with concomitant fusion of each fold at region(s) of opposing mucosal tissue contact. Such injury and/or removal of mucosal tissue may, for example, be achieved via electromagnetic, thermal, chemical, biologic or mechanical modalities, or a combination thereof. Additional injury/removal modalities will be apparent to those of skill in the art.

During the period of time necessary for remodeling and/or scar tissue formation, securing elements, such as those previously described, may be utilized to maintain contact of opposing injured tissue along each tissue fold. The securing elements optionally may be removed or may biodegrade upon formation of scar tissue. Alternatively, the elements may be left in place indefinitely to reinforce the scar tissue and facilitate prolonged maintenance of the tissue folds. However, upon formation of the scar tissue, the securing elements need no longer solely maintain the folds.

FIG. 5 illustrates healing and remodeling of the approximated tissue folds along the regions of contact M between approximated tissue folds F₁ and F₂. By injuring, and preferably removing, mucosal tissue along the contact regions, the folds have fused together. Such injury and/or removal of mucosal tissue may be achieved before, during and/or after approximation of folds F₁ and F₂. By healing and fusing together, the approximated folds may no longer be solely dependent on additional securing element(s), for example, anchor assembly 80 of FIG. 4, to maintain their approximation.

In FIG. 5, the securing element(s) illustratively have been removed or have biodegraded from the tissue folds, and approximation of the folds is solely maintained via tissue remodeling/scar tissue formation. However, it should be understood that securing element(s) alternatively may be left in place to reinforce the remodeled scar tissue that fuses folds F₁ and F₂. In this manner, the securing element(s) and the scar tissue may share the burden of maintaining approximation and may also act as redundant safety mechanisms to maintain tissue fold approximation, should either the remodeled tissue or the securing element(s) fail.

The method of FIG. 5 may, for example, comprise the steps of forming at least two tissue folds at a target tissue region, injuring or removing tissue disposed between the at least two tissue folds, and approximating the at least two tissue folds such that the at least two tissue folds contact one another in a region of injured or removed tissue, thereby promoting healing between the at least two tissue folds. The method may also comprise providing a shape-lockable device, endoluminally advancing the shape-lockable device to a target tissue region, then shape-locking the device and advancing instruments along or through the shape-lockable device to form the tissue folds. The instruments/tools alternatively may be coupled to, or integrated into, the shape-lockable device.

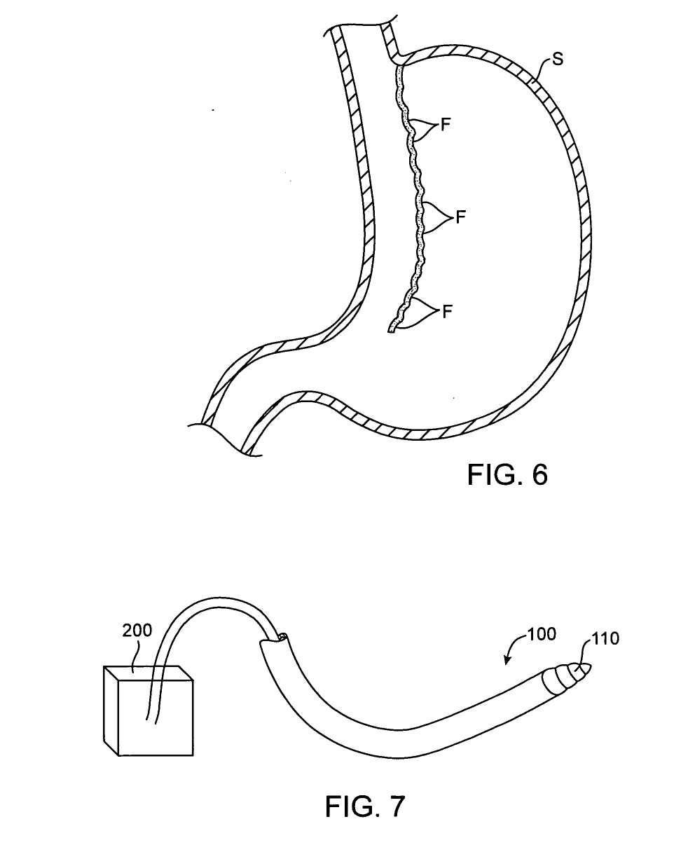

Referring now to FIG. 6, a method of utilizing the remodeling technique of FIG. 5 to perform an illustrative medical procedure is described. In FIG. 6, stomach S comprises a series of adjacent approximated and remodeled tissue folds F that partition the stomach into first and second lumens over at least a portion of its length. The partitioned stomach is expected to restrict or reduce the passage of food therethrough, thereby promoting weight loss in obese or morbidly obese patients. Additional medical procedures utilizing apparatus and methods of the present invention, such as the treatment of gastroesophageal reflux disease (“G.E.R.D.”), will be apparent.

The method of FIG. 6 may comprise the steps of forming anterior and posterior tissue folds within a patient's stomach in a vicinity of the patient's gastroesophageal junction, injuring, and preferably removing, tissue disposed between the anterior and posterior tissue folds, approximating the anterior and posterior tissue folds such that the approximated tissue folds contact one another in a region of injured and/or removed tissue, thereby promoting healing between the at least two tissue folds, and repeating the method at at least one adjacent location within the patient's stomach to partition and reduce the patient's stomach. The method preferably is performed endoluminally, but additionally or alternatively may be performed per-orally, transgastrically, laparoscopically, intraluminally, etc.

With reference to FIG. 7, an injury tool for locally injuring or removing mucosal tissue to promote healing is described. In a first variation, injury tool 100 comprises ablation element 110. Ablation element 110 may, for example, comprise a monopolar or bipolar Radio Frequency (“RF”) ablation element, and illustratively is proximally coupled to RF generator 200 for energizing the ablation element. Element 110 may be brought into contact with mucosal tissue M of FIGS. 4 and 5, and then energized to ablate or otherwise injure and/or remove the mucosal tissue to initiate a wound healing response for fusing the approximated folds together. Ablation element 110 of injury tool 100 may, for example, comprise a commercially available RF probe, such as the Gold Probe™ Electrohemostasis Catheter from the Boston Scientific Corporation of Natick, Mass.

Injury tool 100 may be used in combination with apparatus for forming, securing and approximating tissue folds. For example, the tool may be integrated into tissue manipulation assembly 14 of FIGS. 1 and 2. Alternatively, the tool may be advanced through body 12 of plication assembly 10. Furthermore, tool 100 optionally may be provided as part of a system of tools, which may, for example, include plication assembly 10 and/or overtube 1.

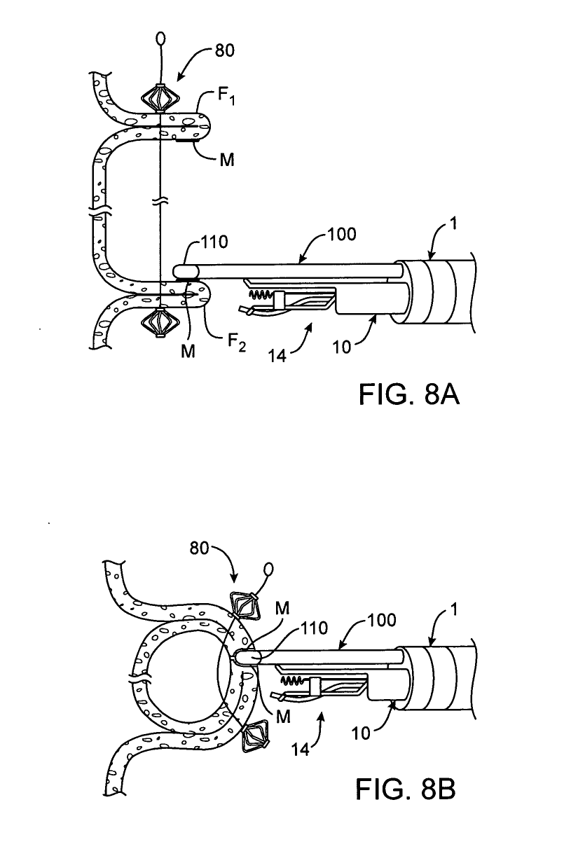

With reference to FIGS. 8, additional methods of utilizing injury tool 100 are described. As seen in FIG. 8A, the tool may be advanced through overtube 1 for injuring mucosal tissue M of tissue folds F. Overtube 1 may, for example, comprise multiple lumens for advancing tool 100 adjacent to assembly 14 as shown. The injured mucosal tissue M will be in contact upon approximation of folds F, which is expected to cause remodeling and fusion of the folds together. FIG. 8B illustrates an alternative method whereby contacting mucosal tissue M is injured after approximation of the folds. Injuring the tissue post-approximation may decrease a risk of injuring the tissue in an improper location.

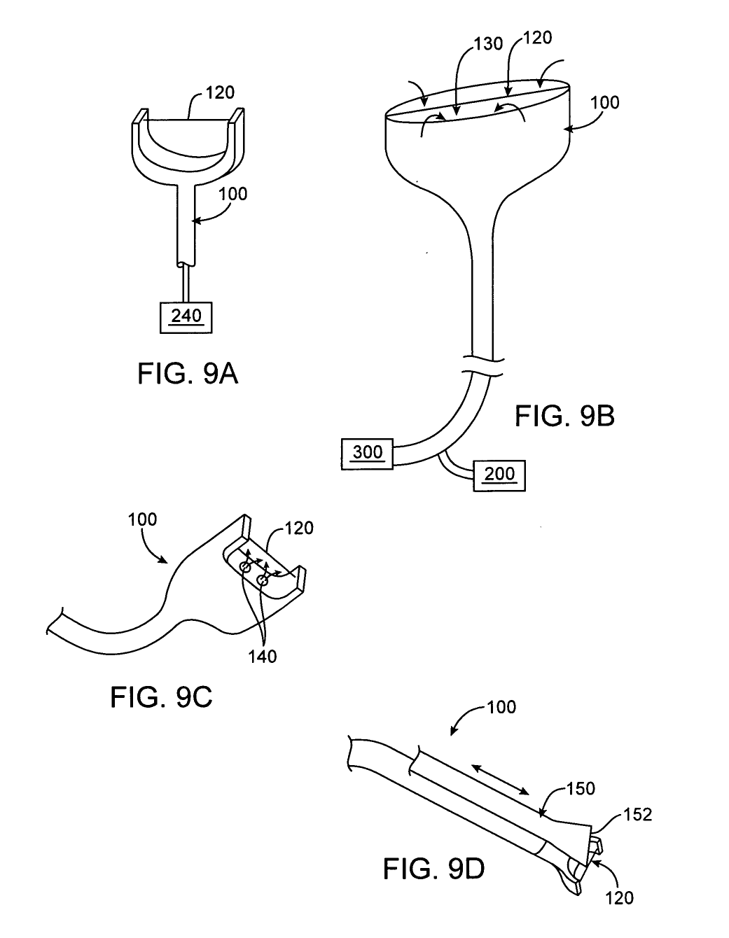

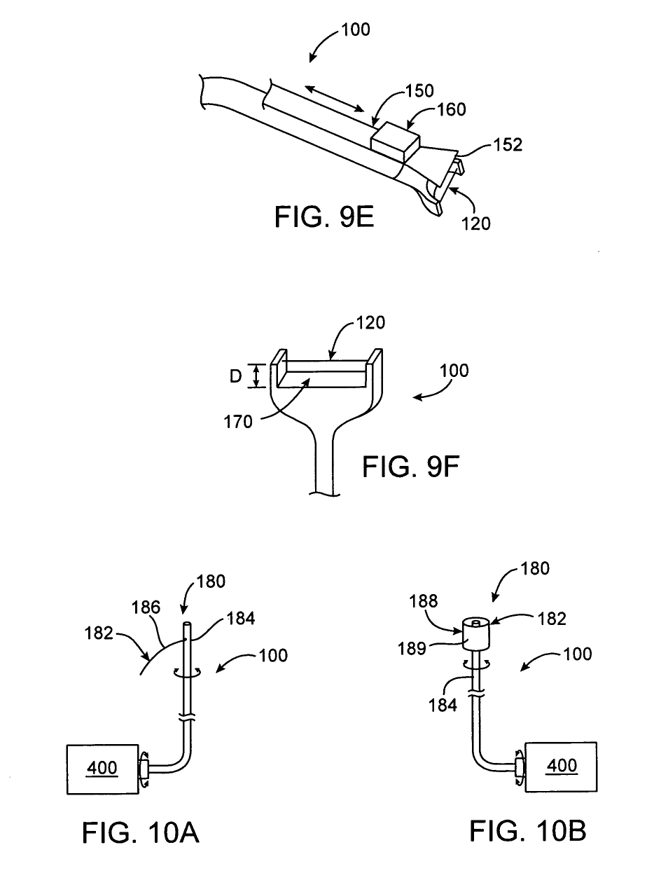

Referring now to FIGS. 9, additional variations of injury tool 100 are described. As seen in FIG. 9A, tool 100 may comprise elongated injury element 120. Element 120 may, for example, comprise an energizable electrode for ablating the mucosa, or may comprise a cutting element for mechanically injuring or removing the mucosa. As yet another alternative, element 120 may comprise both an energizable electrode and a cutting element. Element 120 may, for example, be about 1 cm (about 0.4 in.) long and about 0.008-0.013 cm (about 0.003-0.005 in.) thick, though any alternative dimensions may be provided. Element 120 illustratively is proximally connected to previously described RF generator 200.

FIG. 9B illustrates another variation of tool 100 and element 120 having suction element 130. Suction element 130 is coupled to suction pump 300. Elements 120 and 130 may be used in combination to remove mucosal tissue. For example, element 120 may ablate or cut the mucosal tissue, while suction drawn through element 130 via pump 300 may remove the ablated or cut tissue. Suction element 130 may also be used to engage tissue prior to injury.

FIG. 9C provides still another variation of tool 100 and element 120 comprising fluid injection element 140. Element 140 may be used to inject saline or other fluids, e.g., for cooling tissue ablated with element 120 and/or to cleanse tissue ablated or cut with the element. Additionally or alternatively, abrasive chemicals may be injected through element 140 to injure the mucosa.

As seen in FIG. 9D, injury tool 100 may comprise removal element 150, which may comprise edge 152 that may be translated relative to injury element 120 to slough off mucosal tissue injured by the element. The sloughed off tissue may be allowed to simply pass, e.g., through the patient's GI system. Alternatively, as seen in FIG. 9E, tool 100 may further comprise capture element or compartment 160 for capturing removed tissue. Removal of injured tissue may accelerate or enhance the wound healing response and/or may increase the tenacity of bonding between contacting tissue surfaces.

FIG. 9F depicts a variation of tool 100 and element 120 comprising depth-limiting element 170. The distance D between injury element 120 and depth-limiting element 170 may be specified to provide a maximum depth to which injury of mucosal tissue may occur. Such a depth-limiting feature may be used to ensure that injury is inflicted upon the tissue only to specified layers, e.g., the mucosal and/or muscularis layers of tissue. For example, distance D may comprise a distance of about 5 mm (about 0.2 in.), though any alternative distance may be provided. When injury element 120 comprises a mechanical cutting element, the maximum depth of injury is expected to approximate distance D. When the injury element comprises an ablation element, the depth will depend upon both distance D and the parameters of ablation energy passed through element 120, as well as characteristics of the tissue being ablated.

Referring now to FIG. 10, additional variations of tool 100 are described comprising mechanical injury element 180having injury tip 182 coupled to rotating shaft 184. Shaft 184 preferably is proximally coupled to motor 400. In FIG. 10A, injury tip 182 comprises elongated element 186 that extends substantially perpendicular to rotating shaft 184. Element 186 preferably comprises a polymeric or metal wire or fiber. When shaft 184 is rotated with significant velocity, mucosal tissue ‘whipped’ by element 186 is injured to initiate the healing response. In FIG. 10B, alternative injury tip 182 comprises burr 188 having rough or abrasive surface 189. When rotated against tissue, the abrasive surface injures the tissue.

Injury tool 100 preferably is used in combination with additional tools, e.g., for forming and securing gastrointestinal tissue folds, such as plication assembly 10 and/or overtube 1. More generally, the additional tools may comprise tools for accessing, visualizing, grasping, maneuvering, piercing, folding, plicating, approximating, securing, characterizing, sampling and/or suturing GI tissue. One or more of the tools optionally may be steerable and/or shape-lockable/rigidizable. Additional tools will be apparent to those of skill in the art. Such tools may be provided as a kit or system in combination with the injury tool. Furthermore, the injury tool may be integrated into multi-functional tools. The tools preferably are configured for endoluminal passage within a patient's GI tract.

Although preferred illustrative embodiments of the present invention are described hereinabove, it will be apparent to those skilled in the art that various changes and modifications may be made thereto without departing from the invention. It is intended in the appended claims to cover all such changes and modifications that fall within the true spirit and scope of the invention.

What is claimed is:

1. Apparatus for achieving prolonged maintenance of gastrointestinal tissue folds, the apparatus comprising an injuring device adapted to injure a region of tissue and remove at least a portion of the injured tissue.

2. The apparatus of claim 1, further comprising a tissue plication device for forming at least two tissue folds to be maintained.

3. The apparatus of claim 2, further comprising a tissue approximation device for approximating the at least two tissue folds such that the at least two tissue folds contact one another along a region of injured tissue, thereby promoting healing between the at least two tissue folds.

4. The apparatus of claim 1, further comprising a shape-lockable device through which the injuring device is adapted to be advanced.

5. The apparatus of claim 1, wherein the injuring device comprises an injury or removal modality chosen from the group consisting of ablation, burning, charring, cutting, abrasion, chemical irritation, biological irritation and combinations thereof.

6. The apparatus of claim 2, wherein the injuring device is integrated into the tissue plication device.

7. The apparatus of claim 3, wherein the injuring device is integrated into the tissue approximation device.

8. The apparatus of claim 1 further comprising a securement device for securing the injured region of tissue.

9. The apparatus of claim 1 further comprising at least one securing element for securing the injured region of tissue.

10. The apparatus of claim 9, wherein the at least one securing element is configured to secure the injured region of tissue.

11. The apparatus of claim 9, wherein the at least one securing element is biodegradable.

12. The apparatus of claim 9, wherein the at least one securing element is configured for removal.

13. The apparatus of claim 9, wherein the at least one securing element is configured to temporarily secure the injured region of tissue.

14. The apparatus of claim 9, wherein the at least one securing element is configured to permanently secure the injured region of tissue.

15. The apparatus of claim 4, wherein the shape-lockable device is steerable.

16. A method for achieving prolonged maintenance of gastrointestinal tissue folds, the method comprising:
injuring at least a first region of tissue to be approximated against a second region of tissue;
removing at least a portion of injured tissue from the first region to be approximated; and
contacting the first region of tissue against the second region of tissue to promote healing between the first and second regions of tissue.

17. The method of claim 16, further comprising injuring the second region of tissue prior to or after contacting the first region of tissue against the second region of tissue.

18. The method of claim 17, further comprising removing at least a portion of injured tissue from the second region to be approximated.

19. The method of claim 17, further comprising approximating the first region and second region of tissue such that the injured tissue contacts one another such that healing between the injured tissue is promoted.

20. The method of claim 16, wherein injuring at least a first region of tissue comprises ablating the tissue.

21. The method of claim 16, wherein injuring at least a first region of tissue comprises abrading the tissue.

22. The method of claim 16, wherein injuring at least a first region of tissue comprises cutting the tissue.

23. The method of claim 16, further comprising securing the first region of tissue to the second region of tissue.

24. The method of claim 23, wherein securing the first region of tissue against the second region of tissue further comprises temporarily securing the tissue folds.

25. The method of claim 16, wherein injuring at least a first region of tissue further comprises controlling a depth of tissue injury.

26. A method for achieving prolonged maintenance of gastrointestinal tissue folds, the method comprising:
endoluminally advancing a device to a target tissue region;
shape-locking the device;
forming at least two tissue folds at the target tissue region with instruments advanced along or coupled to the device;
injuring or removing tissue disposed between the at least two tissue folds; and
approximating the at least two tissue folds such that the at least two tissue folds contact one another in a region of injured or removed tissue, thereby promoting healing between the at least two tissue folds.

27. The method of claim 26, wherein injuring or removing tissue comprises ablating the tissue.

28. The method of claim 26, wherein injuring or removing tissue comprises abrading the tissue.

29. The method of claim 26, wherein injuring or removing tissue comprises cutting the tissue.

30. The method of claim 26, further comprising capturing and removing injured tissue.

31. The method of claim 26, further comprising securing the approximated tissue folds together.

32. The method of claim 31, wherein securing the approximated tissue folds together further comprises temporarily securing the approximated tissue folds.

33. The method of claim 26, wherein injuring or removing tissue further comprises controlling a depth of tissue injury or removal.

34. A method for performing gastric reduction, the method comprising:
forming anterior and posterior tissue folds within a patient's stomach in a vicinity of the patient's gastroesophageal junction;
injuring or removing tissue disposed between the anterior and posterior tissue folds;
approximating the anterior and posterior tissue folds such that the approximated tissue folds contact one another in a region of injured or removed tissue, thereby promoting healing between the at least two tissue folds; and
repeating the method at at least one adjacent location within the patient's stomach to reduce the patient's stomach.

35. The method of claim 34, wherein the method is performed endoluminally.

36. A system of tools for achieving prolonged maintenance of gastrointestinal tissue folds, the apparatus comprising:
a shape-lockable access tool;
a tool for forming tissue folds;
a tool for injuring or removing tissue disposed between tissue folds; and
a tool for approximating tissue folds.

37. The system of tools of claim 36 further comprising a tool for securing tissue folds.

38. Apparatus for achieving prolonged maintenance of gastrointestinal tissue folds, the apparatus comprising:
a device for forming at least two tissue folds;
a device for injuring or removing tissue disposed between the at least two tissue folds; and
a device for approximating the at least two tissue folds such that the at least two tissue folds contact one another in a region of injured or removed tissue, thereby promoting healing between the at least two tissue folds,
wherein the device for injuring or removing tissue comprises an element for controlling a depth of tissue injury or removal.
